(19) United States
(12) Patent Application Publication
(10) Pub. No.: US 2025/0221858 A1
(43) Pub. Date: Jul. 10, 2025

(54) COUPLING ARRANGEMENT FOR GOGGLES

(71) Applicant: IMPERTECH INDUSTRIES Ltd., Barkan (IL)

(72) Inventor: Israel GURMAN, Rehovot (IL)

(21) Appl. No.: 18/726,231

(22) PCT Filed: Dec. 27, 2022
(86) PCT No.: PCT/IL2022/051395

§ 371 Date: Jul. 2, 2024

(30) Foreign Application Priority Data
Jan. 2, 2022 (IL) 289566

(51) Int. Cl. A61F 9/02

(52) U.S. Cl. CPC A61F 9/029

(57) ABSTRACT

A coupling arrangement for goggles, comprising a body member having a central opening and bounded by a peripheral element with a continuous channel, monolithic goggles having a peripheral edge shaped as the peripheral element and is receivable in the channel to occlude the central opening, at least one compression element interacting with the peripheral element to urge the peripheral edge of the goggles in engagement with a surface of the channel, and at least one anchoring element protruding from the goggles and coupled with the at least one compression element that applies a clamping force onto the at least one compression element, wherein the applied clamping force is transmitted along a corresponding region of the peripheral element to resist movement of the goggles therein and to prevent infiltration of a fluid across the peripheral edge of the goggles towards the face of a wearer of the goggles.

### FIELD OF THE INVENTION

The present invention relates to the field of protective eyewear. More particularly, the invention relates to a coupling arrangement for goggles.

### BACKGROUND OF THE INVENTION

Many different types of goggles for protecting the eyes of athletes and workers are in use. Some types of goggles such as swimming goggles and blowtorch goggles cover only the eyes, while others such as scuba diving goggles also cover the nose and half of the face, also being considered as a mask. In addition to the size of the goggles, the composition and structure of the goggle lenses differ from one goggle to another, some such as welding goggles being equipped with a filter to protect against viewing the generated ultraviolet light while others such as power tool goggles are impact resistant to protect the eyes from flying metal and plastic and also have ventilation features.

Each goggle type needs a dedicated frame to hold the lenses. A user having several types of goggles therefore has to allocate sufficient storage space to allow each type to be readily accessible prior to use.

Goggles for masks, for example gas masks, have to be sealingly engaged against the skin of the user wearing the goggles to prevent infiltration to the eyes of a surrounding fluid that is known to irritate or otherwise damage the eyes. A significant challenge facing goggle designers is the ability to cost effectively hold the goggles while ensuring that they are suitably sealed, particularly when they provide a panoramic field of vision.

Some goggles are connected peripherally to the frame by adhesion or welding. Stress often develops in such peripheral connections due to head movements of the goggle wearer, such that, over the course of time, microscopic or larger sized openings are formed in the peripheral connections or alternatively goggle elements adapted to be in sealing engagement with the skin of the wearer become spaced therefrom. These unwanted changes caused by expansion forces compromise the protective sealing of the goggles.

Many goggles are configured with two integral lens portions that each protectively enclose a corresponding eye of the goggle wearer and with an intermediate portion continuous with, but of a significantly smaller height to accommodate the presence of the nose bridge than, the two lens portions. Due to the non-uniform peripheral shape of the goggles, a securing element adapted to apply a radially inward force to resist an unwanted expansion force applied by the goggle wearer cannot be readily connected to the entire periphery of a lens portion.

It is an object of the present invention to provide elements of protective goggles that are ensured of remaining in sealing engagement with the face of the goggle wearer to prevent infiltration to the eyes of a surrounding fluid that is potentially irritating or damaging.

It is an object of the present invention to provide a coupling arrangement for goggles that resists an unwanted expansion force applied by the goggle wearer that would compromise the protective sealing of the goggles.

It is an additional object of the present invention to provide eyewear with means for speedily replacing a first goggle type with a second goggle type when desired, to minimize the space occupied by the various frames.

Other objects and advantages of the invention will become apparent as the description proceeds.

### SUMMARY OF THE INVENTION

A coupling arrangement for goggles comprises a body member configured with a central opening that is bounded by a peripheral element which is configured with a continuous channel, monolithic goggles having a peripheral edge of a same shape as the shape of said peripheral element and that is receivable in said channel to occlude said central opening, at least one compression inducing element interacting with said peripheral element, and at least one anchoring element protruding from said goggles and coupled with said at least one compression inducing element that applies a clamping force onto said at least one compression inducing element, wherein the applied clamping force is transmitted along a corresponding region of said peripheral element to resist movement of said goggles at said corresponding region and to urge the peripheral edge of said goggles in engagement with a surface of said channel to prevent infiltration of a surrounding fluid across said peripheral edge of the goggles to the eyes or nostrils of a wearer of the goggles.

In one aspect, one or two of the at least one anchoring element divides the goggles into different goggle regions, each of said goggle regions being associated with the corresponding peripheral element region along which the applied clamping force is transmitted.

In one aspect, the at least one anchoring element is coupled with the at least one compression inducing element in such a way that a posteroanterior directed clamping force is applied.

In one embodiment, the at least one compression inducing element comprises an upper rim-fastener and a lower rim-fastener that are interconnected.

To maintain the peripheral edge of the goggles in sealing engagement with a surface of the channel, the goggles are generally coupled with the lower rim-fastener, such as by one of the at least one anchoring elements that protrudes, for example protruds perpendicularly, from a rib provided with the goggles and that is releasably introduced through an aperture formed in a mounting element of the lower rim-fastener.

The rib may be provided at a centerline of the goggles that may coincide with a nose bridge portion.

The goggles may be additionally coupled with the upper rim-fastener, such as by an additional anchoring element of the at least one anchoring elements that protrudes from the rib and that is releasably introduced through an aperture formed in a mounting element of the upper rim-fastener.

In one aspect, the central opening of the body member is completely bounded by the peripheral element.

In one aspect, the body member is sealingly engageable with the face of the wearer. The body member may for example be made of elastomeric material or hard plastic material with an elastomeric backing that is sealingly engageable with the face of the wearer.

In one aspect, the peripheral element of the body member is configured with an inner surface that is sealingly engageable with the face of the wearer, an outer surface and a base that extends from the inner surface to the outer surface to define the channel.

In one aspect, the at least one compression inducing element is configured to urge the peripheral edge of the goggles in engagement with the base of the channel.

In one aspect, the peripheral edge of the goggles is frictionally receivable within the channel.

In one aspect, the at least one compression inducing element releasably interacts with the peripheral element.

In one aspect, the at least one compression inducing element irreleasably interacts with the peripheral element.

Another aspect of the invention is related to an eyewear assembly that comprises the coupling arrangement.

In one aspect, the body member comprises a mask appendage.

In one aspect, the eyewear assembly further comprises securing means for securement to the head of the wearer.

In one aspect, the goggles are detachable from the body member and are replaceable with another type of goggles.

### BRIEF DESCRIPTION OF THE DRAWINGS

In the drawings:

FIG. 1 is a perspective view of an eyewear assembly according to one embodiment;

FIG. 2 is an exploded view of the eyewear assembly of FIG. 1;

FIG. 3 is a cross sectional perspective view of the eyewear assembly of FIG. 1 when cut along a medial portion thereof, showing a coupling arrangement therefor;

FIG. 4 is a perspective view of an assembled gas mask that is equipped with the coupling arrangement of FIG. 3;

FIG. 5 is an exploded view of the gas mask of FIG. 4;

FIG. 6 is a perspective view of an assembled gas mask that is equipped with a coupling arrangement according to another embodiment; and

FIG. 7 is a cross sectional perspective view of a portion of the gas mask of FIG. 6 when cut along a medial portion thereof, showing the coupling arrangement therefor.

### DETAILED DESCRIPTION OF THE INVENTION

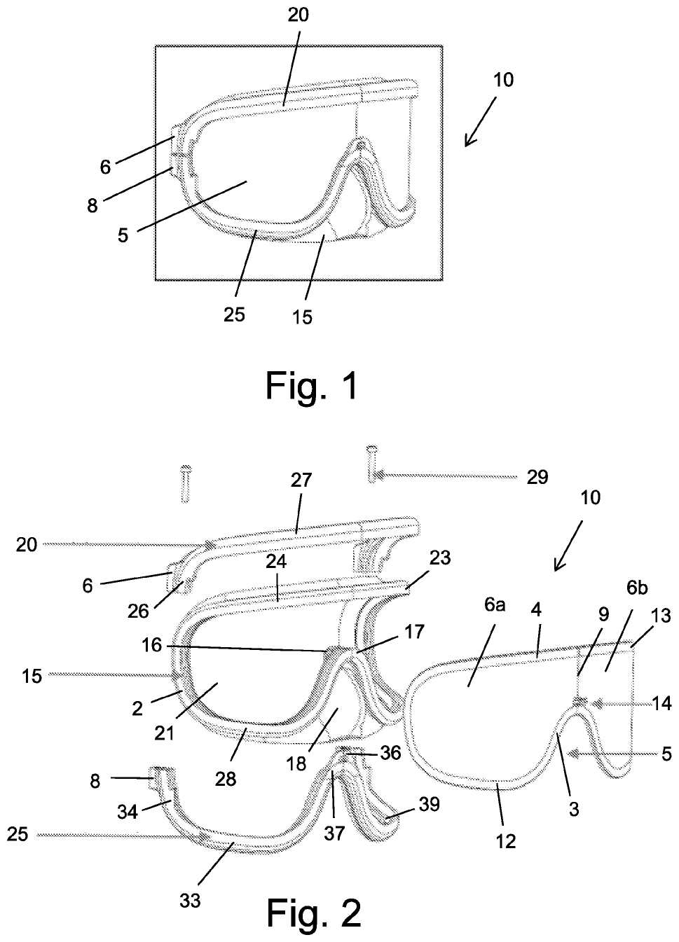

FIG. 1 illustrates eyewear assembly 10 according to one embodiment that is configured with detachable or replaceable protective goggles 5 and with a coupling arrangement that facilitates detachable coupling of goggles 5 to eyewear assembly 10 while maintaining a sealing engagement to the face of the wearer to prevent passage of potentially damaging matter, such as sea water or noxious gas, through an unwanted clearance to the eyes or nostrils of the user. When coupled by the coupling arrangement as shown, goggles 5 are fitted in body member 15 and are also securely retained by upper rim-fastener 20 and lower rim-fastener 25, which are connected to each other by adjacent fittings 6 and 8, respectively, to resist goggle detachment.

The directional terms mentioned herein such as “upper”, “lower”, “vertical” and “lateral” are described in relation to a regular standing or sitting orientation of the wearer, but it will be appreciated that the invention is likewise applicable to any other orientation of the wearer, mutatis mutandis.

FIG. 2 illustrates an exploded view of eyewear assembly 10.

The shape of protective goggles 5 is delimited by peripheral goggle border 13 that includes an upper elongated goggle perimeter 4, two lower convex portions 12, and a lower inverted U-shaped nose bridge 3 that extends continuously from the two lower convex portions 12. Goggles 5 are configured with lens portions 6a and 6b which are made of a transparent or translucent material having a specific composition and structure being suitable to provide desired protection, and are integrally formed together as an integral monolithic element. A planar rib 9, which divides goggles 5 into the two symmetric lens portions 6a and 6b, coincides with the centerline of goggles 5, vertically extending between the summit of nose bridge 3 and upper goggle perimeter 4 and having a length significantly shorter than the height of lens portions 6a and 6b. Each of lens portions 6a and 6b is slightly outwardly convex and has an oval projection when viewed frontwardly to protectively enclose a corresponding eye of the goggle wearer. An anchoring element 14 protrudes perpendicularly from rib 9 directly above nose bridge 3.

The regions of lens portions 6a and 6b slightly laterally spaced from rib 9 gradually form a convex shape. The convexity of lens portions 6a and 6b is generally sufficiently great to ensure an unobstructed view to the side. While the vertical distance of the two lens portions is substantially uniform at a corresponding lateral terminal end 2, the vertical distance of each lens portion between goggle perimeter 4 and a corresponding lower convex portion 12 changes until approaching rib 9. Goggle border 13, which may not be made of a transparent or translucent material, peripherally extends throughout outer edges of lens portions 6a and 6b.

Rib 9 may be made of the same material and of the same thickness as the lens portions 6a and 6b. Alternatively, rib 9 may be made of a different material or of a different thickness than lens portions 6a and 6b. It may be imparted with a special rigidity that produces tension or another feature that induces improved sealing engagement together with anchoring element 14.

In other embodiments, goggles 5 may be fabricated as a single plastic sheet, but with the interaction of rib 9, anchoring element 14 and the lower rim-fastener, as will be described hereinafter, the goggles are able to be urged to assume a desired convex configuration.

It will be appreciated that any other suitable goggle configuration is also in the scope of the invention, insofar as the goggles are a one-piece unit.

Body member 15 has a central opening 21 that is bounded by a continuous peripheral element 23 of the same shape and curvature as corresponding regions of goggle border 13, including upper elongated portion 24 and two lower convex portions 28, although of slightly larger dimensions than goggle border 13.

Body member 15 may be made of elastomeric material that is sealingly engagaeable with the face of the wearer, and may be further configured with nose-receiving appendage 18 extending downwardly from, and considerably thinner than, nose bridge 17, for use in conjunction with a mask, such as a gas mask. It may be appreciated that body member 15 may be made of hard plastic material with an elastomeric backing to provide the sealing engagement. Likewise, body member 15 may be provided without the mask appendage, while being secured to the head of the wearer by various means such as temples or straps.

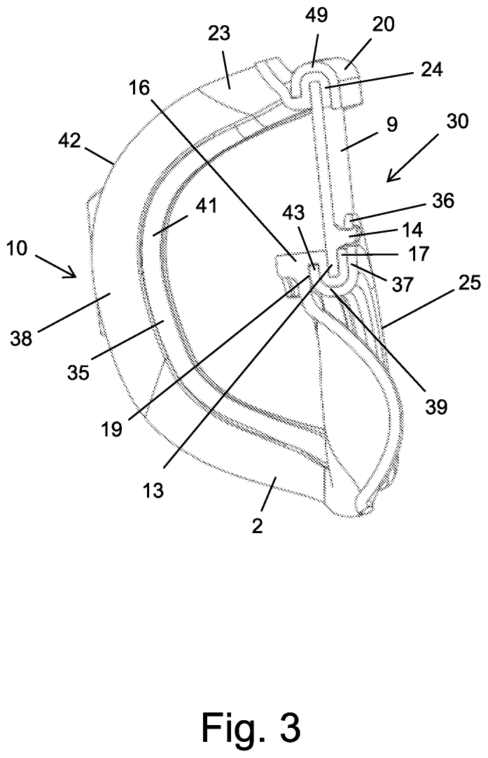

As shown in FIG. 3, peripheral element 23 is configured with an integral inner surface 38, outer surface 41, and base 42 disposed between the outer and inner surfaces to define a continuous channel 35. Channel 35 is configured such that inner surface 38 and outer surface 41 protrude from base 42 by a same dimension which is slightly greater than the width of goggle border 13 and that the gap of the channel corresponding to the distance between the outer surface and inner surface and to the width of the base is substantially equal to, or slightly greater than, the thickness of goggle border 13. Base 42 may be concave as shown, or alternatively may be planar, such as perpendicular to surfaces 38 and 41, or oblique thereto. The dimensions of channel 35 may be uniform throughout peripheral element 23, or alternatively may be different from one region to another. Similarly, the dimensions of goggle border 13 may be uniform, or alternatively may be different from one region to another in order to be frictionally fit within channel 35. A support element 16 extends rearwardly from peripheral element 23 at the summit of body member nose bridge 17.

With reference also to FIG. 2, upper rim-fastener 20 has an elongated portion 27 adapted to be engaged with, and cover, the upper elongated portion 24 of the peripheral element 23 of body member 15. Elongated portion 27 may be configured with a concave body member engaging surface 49 that defines an engagement cavity within which is receivable a concave peripheral element base 42. Upper rim-fastener 20 is generally also configured with two downwardly curved portions 26 that each continuously and smoothly curves downwardly from elongated portion 27 and is adapted to be engaged with a corresponding upper lateral end portion of the body member. The downwardly curved portions 26 may be configured with an engagement cavity, or alternatively with a recessed body member engaging surface that is sufficient to resist disengagement from the body member. Hollow fitting 6, in which is received a tightening screw 29 for interconnecting upper rim-fastener 20 and lower rim-fastener 25, is formed integrally with, and positioned outwardly from, corresponding downwardly curved portion 26.

Lower rim-fastener 25 has two concave portions 33 each adapted to be engaged with, and cover, a corresponding lower convex portion 28 of the body member peripheral element 23. Concave portion 33 may be configured with a concave body member engaging surface 39 that defines an engagement cavity within which is receivable a concave peripheral element base 42. Also, an upwardly curved portion 34 continuously and smoothly curves upwardly from a corresponding concave portion 33 and is adapted to be engaged with a corresponding lower lateral end portion of the body member, such as by an engagement cavity or a recessed body member engaging surface. Each concave portion 33 extends continuously to inverted U-shaped nose bridge element 37. A continuous body member engaging surface 39 terminating with a protruding lip extends rearwardly from portions 33 and 34, following the curvature of lower rim-fastener 25. A thin mounting element 36 formed with an aperture protrudes upwardly from the summit of nose bridge 37. Hollow fitting 8, adapted to be aligned with fitting 6, is formed integrally with, and positioned outwardly from, the terminal end of a corresponding upwardly curved portion 34.

FIG. 3 illustrates an embodiment of a coupling arrangement 30 for use in conjunction with eyewear assembly 10 that facilitates a good sealing action while allowing detachment of the goggles.

Coupling arrangement 30 includes continuous channel 35 provided with peripheral element 23 of the body member, which is adapted to receive goggle border 13 throughout the periphery of the latter. Channel 35 is sized such that the distance between its inner surface 38 adapted to contact the face of the goggle wearer and its outer surface 41 is substantially equal to the thickness of the goggle border, allowing the entire periphery of the goggle border to be engaged by a snug frictional fit within the channel 35. Eyewear assembly 10 therefore provides two means of protection against infiltration to the eyes or nostrils of a surrounding fluid that is potentially irritating or damaging-the first being the sealing engagement of the body member inner surface 38 with the face of the wearer and the second being the sealing engagement between the peripheral element base 42 and the edge of goggle border 13, and possibly between the face of goggle border 13 and the inner and outer surfaces of peripheral element 23.

Particularly when the body member is made of resilient elastomeric material such as rubber, but also when it is made of a plastic material such as flexible PVC, at least one region of the body member is liable to become disengaged from the face of the wearer, or the goggle border is liable to become separated from peripheral element base, to compromise the sealing action following head movements.

Such unwanted disengagement is mitigated, or altogether eliminated, when upper rim-fastener 20 and lower rim-fastener 25 are set in engagement with the body member fitted with the goggles and then coupled to each other. The compressive force applied by the upper rim-fastener and lower rim-fastener, which are generally made of a hard plastic such as polycarbonate, in opposite directions forces goggle border 13 to remain in engagement with the peripheral element base 42, thereby resisting infiltration of surrounding fluid. Also, the applied compressive force increases the stiffness of the body member to resist unwanted body member movement.

Even though the body member is rendered stiff by the upper and lower rim-fasteners, it nevertheless has sufficient flexibility to be adjusted in order to compensate for different facial structures of wearers.

One body member region at which the goggle border is liable to become disengaged is the nose bridge element. While lower rim-fastener 25 applies a radially inward compressive force throughout each of its long concave body member engaging surfaces onto the corresponding goggle border region in cooperation with the compressive force applied by upper rim-fastener 20 to ensure continued engagement between goggle border 13 and peripheral element 23, the radially inward compressive force is not appreciably influential in the vicinity of nose bridge portion 17. Unwanted disengagement of the goggle border from the peripheral element in the vicinity of nose bridge portion 17, such as in a lateral direction, is therefore liable to result from head movements by the wearer.

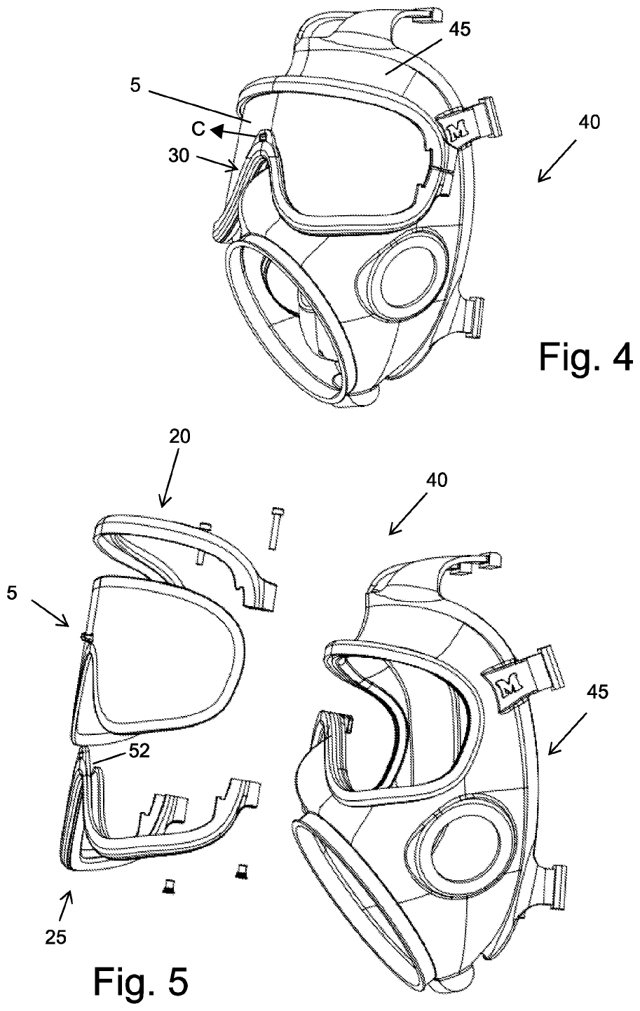

Coupling arrangement 30 advantageously provides the goggles with additional stability while being in sealing engagement with the body member by means of anchoring element 14, which protrudes from rib 9 of the goggles and is introduced through the aperture formed in mounting element 36 of lower rim-fastener 25, to couple the goggles with lower rim-fastener 25. Through the connection of anchoring element 14 and mounting element 36, a clamping force C (FIG. 4), which may be posteroanteriorly directed, is applied onto mounting element 36, and the clamping force is transmitted peripherally along the border element to increase the sealing engagement with the body member and to resist infiltration of the surrounding fluid. Support element 16 that extends rearwardly from body member nose bridge 17 and that may have a rectangular cross section increases the structural strength of eyewear assembly 10 at the connection between anchoring element 14 and mounting element 36. To facilitate engagement of lower rim-fastener 25 in the vicinity of nose bridge 17, a vertical slit 19 is formed in the lower face of support element 16.

The coupling action may be effected when lower rim-fastener 25 is made of flexible plastic material. Once goggle border 13 is fitted within the channel of peripheral element 23, including the illustrated upper elongated portion 24, the lateral end portions 2 and the nose bridge portion 17, lower rim-fastener 25 is displaced upwardly after being aligned with the lower portions of peripheral element 23. When lower rim-fastener 25 is being displaced upwardly, the thin mounting element 36 is flexed to allow the terminal end 43 of body member engaging surface 39 at nose bridge element 37 to be introduced into support element slit 19 without any interference caused by anchoring element 14. After the lower portions of peripheral element 23 including body member nose bridge 17 are received in the engagement cavity 52 (FIG. 5) of lower rim-fastener 25, mounting element 36 is released to allow anchoring element 14 to be introduced into its aperture.

Alternatively, the coupling action may be effected by causing anchoring element 14 to be introduced through the aperture formed in mounting element 36 and then swinging the lower rim-fastener 25 about the pivot point defined by anchoring element 14 until the terminal end 43 of body member engaging surface 39 at nose bridge element 37 is introduced into support element slit 19 and the lower portions of peripheral element 23 are received in the engagement cavity of lower rim-fastener 25.

In other embodiments, anchoring element 14 may be formed at the medial centerline of the goggles without use of a rib, insofar as anchoring element is alignable with the aperture formed in mounting element 36.

Following suitable positioning of lower rim-fastener 25, goggle nose bridge 3, body member nose bridge 17 and rim-fastener nose bridge 37 are aligned, as shown in FIG. 1.

Afterwards, upper rim-fastener 20 is lowered onto the upper portions of peripheral element 23 until the body member engaging surface 49 of upper rim-fastener 20 covers and engages the elongated portion 24 of peripheral element 23. When upper rim-fastener 20 and lower rim-fastener 25 are interconnected such as with tightening screws, a compressive force is applied that forces goggle border 13 to remain in engagement with the peripheral element base.

Coupling arrangement 30 consequently facilitates superior sealing of the goggles in a way that cannot achieved by prior art devices.

Since the goggles are not fixedly connected to the upper and lower rim-fasteners, they are able to be easily detached from the body member in order to replace one goggle type with a second goggle type when desired by performing the simple steps enumerated above in opposite order. For example scuba diving goggles may be replaced with other mask-equipped goggles, such as gas mask goggles. Alternatively, temple-equipped goggles may be replaced with another, such as welding goggles with blowtorch goggles, or mask-equipped goggles may be replaced with temple-equipped goggles.

FIGS. 4 and 5 illustrate a gas mask 40 configured with coupling arrangement 30. Goggles 5 are fitted in the body member 45 which is integrally formed with gas mask 40.

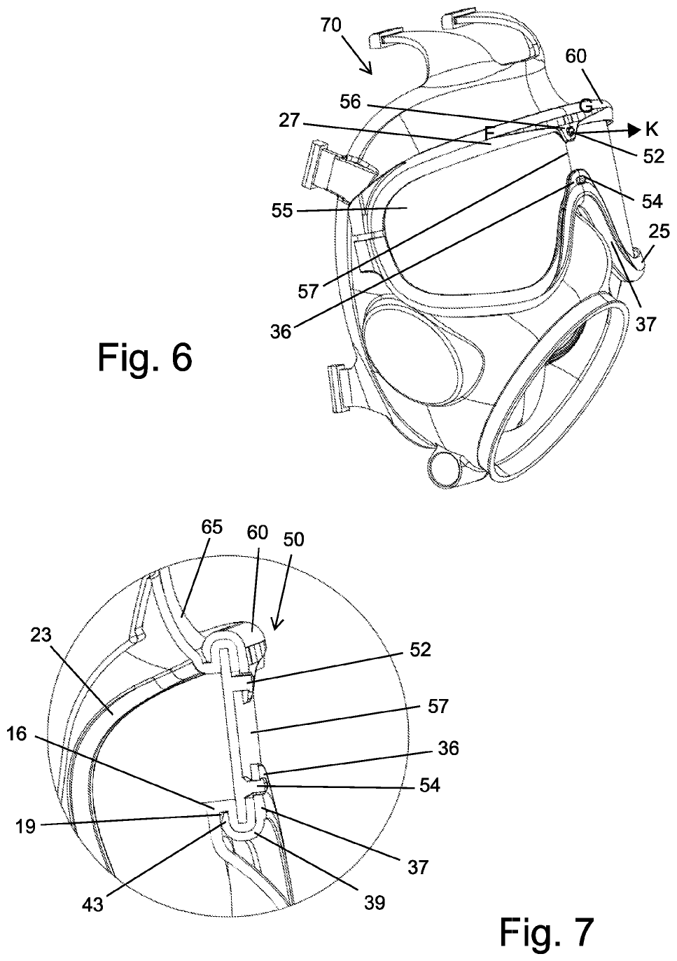

FIGS. 6 and 7 illustrate another embodiment of a coupling arrangement 50 which is provided with two vertically spaced and aligned anchoring elements 52 and 54 that protrude, for example extend substantially perpendicularly, from the medial centerline 57 of goggles 55, such as one configured with a rigid rib. Anchoring elements 52 and 54 are releasably introducible through an aperture formed in the mounting element 56 of the upper rim-fastener 60 and the mounting element 36 of the lower rim-fastener 25, respectively.

Coupling arrangement 50 provided with anchoring elements 52 and 54 is accordingly able to resist disengagement of goggles 55 from body member 65 which is integrally formed with gas mask 70, and additionally facilitates transmission of tension throughout the border element of goggles 55 to increase the sealing engagement of the goggles with body member 65.

As to coupled lower anchoring element 54, as occurring similarly with respect to anchoring element 14 of coupling arrangement 30, mounting element 36 of lower rim-fastener 25 is clamped to goggle medial centerline 57 when terminal end 43 of body member engaging surface 39 at nose bridge element 37 is received in slit 19 of support element 16. The resulting clamping force is transmitted to each curved portion of U-shaped nose bridge element 37 of lower rim-fastener 25, which in turn has a laterally extending component that resists lateral movement of goggles 55 and also provides sealing engagement in a similar direction. This lateral movement resistance is in addition to the vertical movement resistance provided by lower rim-fastener 25, ensuring that goggles 55 will remain in sealing engagement with the peripheral element 23 of body member 65 during normal usage without application of unexpected or excessive force.

Elongated portion 27 of upper rim-fastener 60 has a relatively long radius of curvature when viewed from above, and the presence of upper anchoring element 52 serves to divide elongated portion 27 into two symmetrical portions that are isolated from each other in terms of force transmission. Coupled upper anchoring element 52 causes mounting element 56 of upper rim-fastener 60 to be clamped to goggle medial centerline 57, when lower anchoring element 54 also applies a clamping force C (FIG. 4). The clamping force K applied by upper anchoring element 52, which may be posteroanteriorly directed, is simultaneously transmitted to each of the two symmetric segments F and G of elongated portion 27. The ability of isolated segments F and G to resist disengagement of upper rim-fastener 60 from the corresponding elongated portion of goggles 55 during normal usage is increased by virtue of their shortened force transmitting length.

It will be appreciated that the coupling arrangement may be configured with more than one set of two vertically spaced and aligned anchoring elements. Each set of two anchoring elements divides goggles 55 into different goggle regions. Each of the goggle regions that is defined by a set of anchoring elements is associated with a corresponding body member peripheral element region. The corresponding body member peripheral element region interacts with a corresponding segment of upper rim-fastener 60 along which the applied clamping force is transmitted to resist disengagement of upper rim-fastener 60 from the corresponding elongated portion of goggles 55.

While some embodiments of the invention have been described by way of illustration, it will be apparent that the invention can be carried out with many modifications, variations and adaptations, and with the use of numerous equivalents or alternative solutions that are within the scope of persons skilled in the art, without exceeding the scope of the claims.

1. A coupling arrangement for goggles, comprising:
a) a body member configured with a central opening that is bounded by a peripheral element which is configured with a continuous channel;
b) monolithic goggles having a peripheral edge of a same shape as the shape of said peripheral element and that is receivable in said channel to occlude said central opening;
c) at least one compression inducing element interacting with said peripheral element to urge the peripheral edge of said goggles in engagement with a surface of said channel; and
d) at least one anchoring element protruding from said goggles and coupled with said at least one compression inducing element that applies a clamping force onto said at least one compression inducing element,
wherein the applied clamping force is transmitted along a corresponding region of said peripheral element to resist movement of said goggles at said corresponding region and to prevent infiltration of a surrounding fluid across said peripheral edge of the goggles to the eyes or nostrils of a wearer of the goggles.

2. The coupling arrangement according to claim 1, wherein the at least one anchoring element is coupled with the at least one compression inducing element in such a way that a posteroanterior directed clamping force is applied.

3. The coupling arrangement according to claim 1, wherein one or two of the at least one anchoring element divides the goggles into different goggle regions, each of said goggle regions being associated with the corresponding peripheral element region along which the applied clamping force is transmitted.

4. The coupling arrangement according to claim 1, wherein the at least one compression inducing element releasably interacts with the peripheral element.

5. The coupling arrangement according to claim 1, wherein the at least one compression inducing element irreleasably interacts with the peripheral element.

6. The coupling arrangement according to claim 1, wherein the at least one compression inducing element comprises an upper rim-fastener and a lower rim-fastener that are interconnected.

7. The coupling arrangement according to claim 6, wherein the goggles are coupled with the lower rim-fastener.

8. The coupling arrangement according to claim 7, wherein one of the at least one anchoring elements protrudes from a rib provided with the goggles and is releasably introduced through an aperture formed in a mounting element of the lower rim-fastener.

9. The coupling arrangement according to claim 8, wherein the rib is provided at a centerline of the goggles.

10. The coupling arrangement according to claim 9, wherein the rib is provided at a centerline of the goggles that coincides with a nose bridge portion.

11. The coupling arrangement according to claim 8, wherein the one anchoring element protrudes perpendicularly from the rib.

12. The coupling arrangement according to claim 8, wherein the goggles are additionally coupled with the upper rim-fastener.

13. The coupling arrangement according to claim 12, wherein an additional anchoring element of the at least one anchoring elements protrudes from the rib and is releasably introduced through an aperture formed in a mounting element of the upper rim-fastener.

14. The coupling arrangement according to claim 1, wherein the central opening of the body member is completely bounded by the peripheral element.

15. The coupling arrangement according to claim 1, wherein the body member is sealingly engageable with the face of the wearer.

16. The coupling arrangement according to claim 15, wherein the body member is made of elastomeric material.

17. The coupling arrangement according to claim 15, wherein the body member is made of hard plastic material with an elastomeric backing that is sealingly engageable with the face of the wearer.

18. The coupling arrangement according to claim 1, wherein the peripheral element of the body member is configured with an inner surface that is sealingly engageable with the face of the wearer, an outer surface and a base that extends from the inner surface to the outer surface to define the channel.

19. The coupling arrangement according to claim 18, wherein the at least one compression inducing element is configured to urge the peripheral edge of the goggles in engagement with the base of the channel.

20. The coupling arrangement according to claim 18, wherein the peripheral edge of the goggles is frictionally receivable within the channel.

21. An eyewear assembly, comprising the coupling arrangement according to claim 1.

22. The eyewear assembly according to claim 21, wherein the body member comprises a mask appendage.

23. The eyewear assembly according to claim 21, further comprising securing means for securement to the head of the wearer.

24. The eyewear assembly according to claim 21, wherein the goggles are detachable from the body member and are replaceable with another type of goggles.
